(19) United States
(12) Patent Application Publication
(10) Pub. No.: US 2010/0113406 A1
(43) Pub. Date: May 6, 2010

(54) USE OF GLUCOCORTICOIDS FOR TREATMENT OF CONGESTIVE HEART FAILURE

(76) Inventors: Johannes J.R. Hermans, Schimmert-Nuth (NL); Jos F.M. Smits, Eijsden (NL); Harry A.J. Struijker-Boudier, Rijckholt (NL); Monica Minnaard-Huiban, Maastricht (NL)

Correspondence Address: DURECT CORPORATION;THOMAS P. MCCRACKEN, 2 RESULTS WAY, CUPERTINO, CA, 95014, US

(21) Appl. No.: 12/449,904

(22) PCT Filed: Mar. 14, 2008
(86) PCT No.: PCT/US2008/003448

§ 371 Date: Sep. 1, 2009

Related U.S. Application Data
(60) Provisional application No. 60/918,370, filed on Mar. 16, 2007.

(51) Int. Cl. A61K 31/56; A61P 9/00

(52) U.S. Cl. 514/178

(57) ABSTRACT

Disclosed are methods comprising administering a dosage form comprising a pharmaceutical carrier and at least one glucocorticoid to a subject suffering from congestive heart failure, wherein the at least one glucocorticoid is present in the dosage form in an amount effective to ameliorate aspects of the congestive heart failure. Also disclosed are related methods and dosage forms.

### BACKGROUND OF THE INVENTION

1. Field of the Invention

The invention relates to methods and dosage forms forms comprising at least one glucocorticoid, wherein the at least one glucocorticoid is administered to a subject in an amount effective to ameliorate aspects of the subject's congestive heart failure.

2. Description of Related Art

Congestive heart failure (CHF) is a life-threatening condition in which the heart can no longer pump enough blood to the rest of the body. CHF usually develops slowly as a chronic, long-term condition, although it can sometimes develop suddenly. CHF is a serious disorder that can reduce life expectancy.

CHF may affect the right side, the left side, or both sides of the heart. As the heart's pumping action is lost, blood may back up into other areas of the body, including the lungs, liver, gastrointestinal tract and extremities. Due to reduced blood flow, some organs of CHF subjects may not receive enough oxygen and nutrients, which damages them and reduces their ability to function properly.

According to the American Heart Association, people 40 and older have a 1 in 5 chance of developing CHF in their lifetime. Nearly 5 million people in the United States, already have CHF. About 550,000 people develop CHF each year. People who have other types of heart and vessel disease are also at risk for CHF.

Symptoms of CHF include but are not limited to: weight gain, swelling of feet and ankles, swelling of the abdomen, pronounced neck veins, loss of appetite, indigestion, nausea and vomiting, shortness of breath with activity, or after lying down for a while, difficulty sleeping, fatigue, weakness, faintness,

Treatment of CHF can include lifestyle modifications and medication. Medications useful in the treatment of CHF comprise: ACE inhibitors such as captopril and enalapril; diuretics such as thiazide, loop diuretics, and potassium-sparing diuretics; digitalis glycosides; angiotensin receptor blockers (ARBs) such as losartan and candesartan; beta-blockers

However, these interventions are insufficient to adequately treat all subjects with CHF. As noted above, a large number of subjects suffer from CHF in the US alone. Improved medications would help to treat subjects with CHF.

Therefore, methods and related dosage forms are needed to address the problems in the art relating to improved treatment of CHF.

### BRIEF SUMMARY OF THE INVENTION

In an aspect, the invention relates to methods comprising: administering to a subject suffering from congestive heart failure a dosage form comprising a pharmaceutical carrier and at least one glucocorticoid in an amount ranging from about 0.0001 mg to about 1000 mg.

In other aspects, the invention relates to methods comprising administering a dosage form comprising a pharmaceutical carrier and at least one glucocorticoid to a subject suffering from congestive heart failure, wherein the at least one glucocorticoid is present in the dosage form in an amount effective to ameliorate aspects of the congestive heart failure.

### BRIEF DESCRIPTION OF THE SEVERAL VIEWS OF THE DRAWING

FIG. 1 shows corticosterone concentration in normal Wistar rat interperidcardial fluid.

FIG. 2 shows corticosterone concentration in normal Wistar rat cardiac tissue.

FIG. 3 shows interstitial collagen deposition in rat cardiac tissue.

FIG. 4 shows echocardiographic data as ratios of values at treatment end/treatment start.

FIG. 5 shows heart rates measured in the right carotid artery.

FIG. 6 shows mean right carotid artery blood pressures.

FIG. 7 shows collagen levels in SHHF rat hearts as a function of time.

FIG. 8 shows left ventricular end diastolic pressures at rest.

FIG. 9 shows heart rates during left ventricular contractility measurements.

FIG. 10 shows left ventricular contractility (pressure development rate).

FIG. 11 shows left ventricular relaxation (−dP/dt).

FIG. 12 shows left ventricular epicardial interstitial collagen levels (Sirius Red).

FIG. 13 shows left ventricular gene expression levels of collagens I and III and BNP.

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules, reference to “a solvent” includes a mixture of two or more such compositions, reference to “an adhesive” includes mixtures of two or more such materials, and the like.

### A. INTRODUCTION

Surprisingly, the inventors have discovered that the problems in the art noted above can be addressed by providing methods that comprise administering to a subject suffering from congestive heart failure a dosage form comprising a pharmaceutical carrier and at least one glucocorticoid in an amount ranging from about 0.0001 mg to about 1000 mg.

Further, and also surprisingly, the inventors have discovered that the problems in the art noted above can be addressed by providing methods that comprise administering a dosage form comprising a pharmaceutical carrier and at least one glucocorticoid to a subject suffering from congestive heart failure, wherein the at least one glucocorticoid is present in the dosage form in an amount effective to ameliorate aspects of the congestive heart failure.

Glucocorticoids (naturally occurring and synthetic) are members of a class of steroid hormones characterized by the ability to bind with the intracellular glucocorticoid receptor (GR), induce nuclear translocation of the GR-glucocorticoid complex and trigger similar effects on gene and protein expression. Their use in successfully treating CHF is surprising because the classical effects of glucocorticoids include, but are not limited to exacerbation of hypertension, hyperglycemia and hyperlipidemias. These are all contributing factors to CHF, so usefulness of glucocortcoids would seem to be contraindicated.

The inventors have discovered that, contrary to conventional thinking, glucocorticoids can, in fact, be used to treat CHF, and its related aspects. For instance, in Example 2, in the established spontaneous hypertensive heart failure (SHHF) rat model of CHF, while mean arterial pressure (MAP), heart rate, and echocardiographic examinations revealed no differences between treatments, left ventricular contractility, cardiac filling rate, left ventricular end diastolic pressure, and left ventricular epicardial collagen area all improved following treatment with corticosterone.

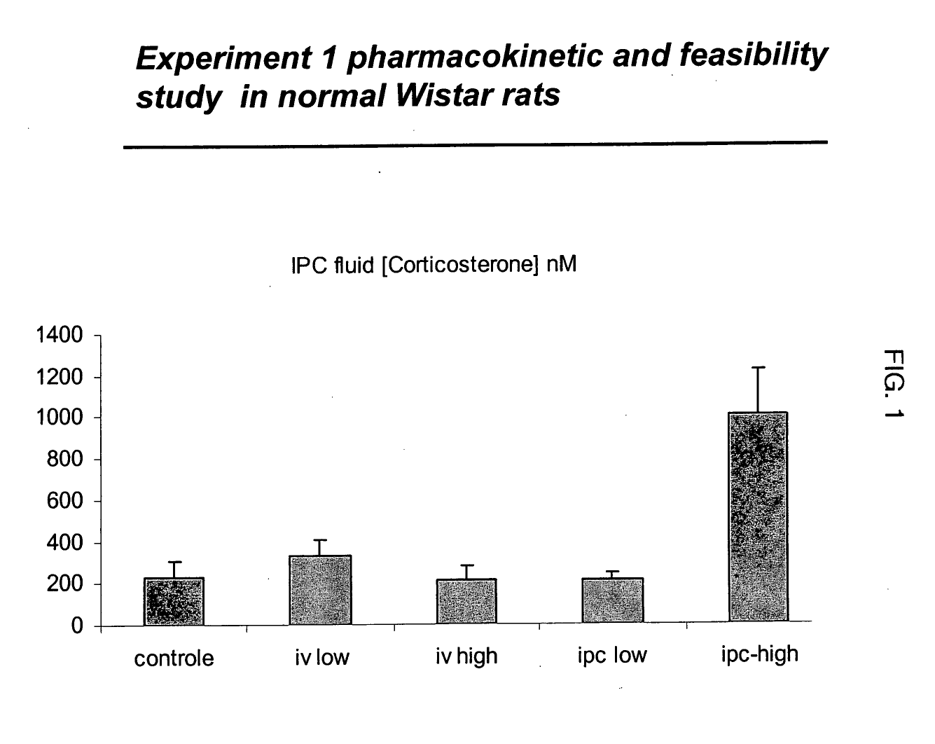

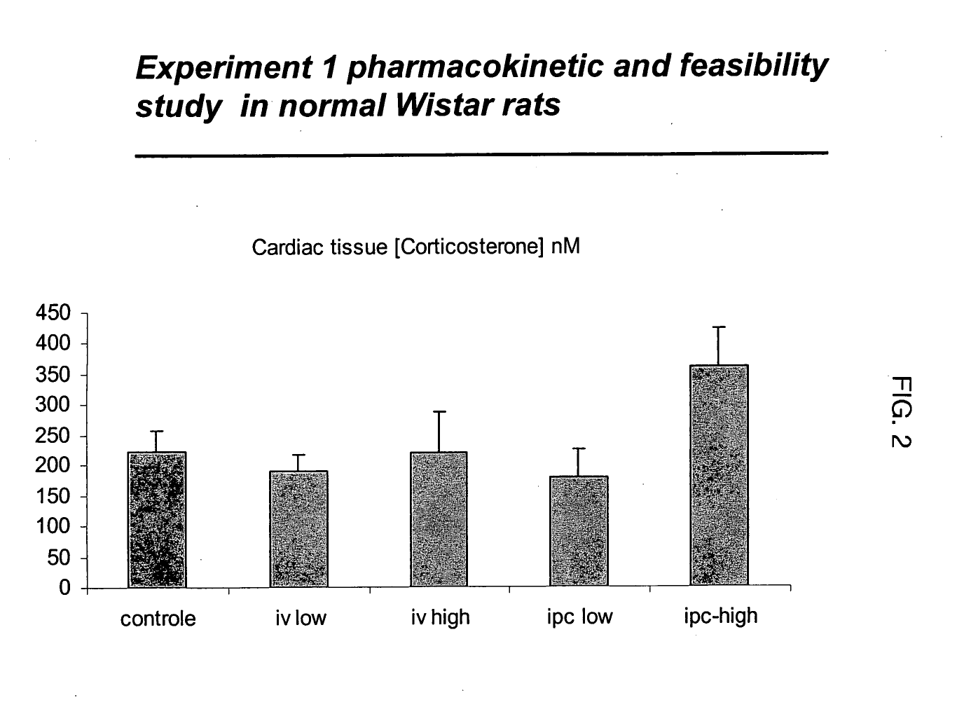

In embodiments, systemic side effects can be reduced by use of local drug administration. As discussed further elsewhere herein, certain forms of local drug administration, such as intrapericardial administration, can provide therapeutic local concentration of drug(s) while reducing the systemic concentration of the drug. Reduced systemic concentration of glucocorticoids, coupled with an effective concentration of at least one glucocorticoid in heart tissue, can provide effective treatment of CHF according to the present invention while reducing the undesirable side effects of the at least one glucocorticoid elsewhere in the body. The results of Example 1, as shown in FIGS. 1 and 2, demonstrate that it is possible to achieve a higher concentration of corticosterone in rat interpericardial fluid and heart tissue using local administration versus that following systemic administration of the same amount of drug.

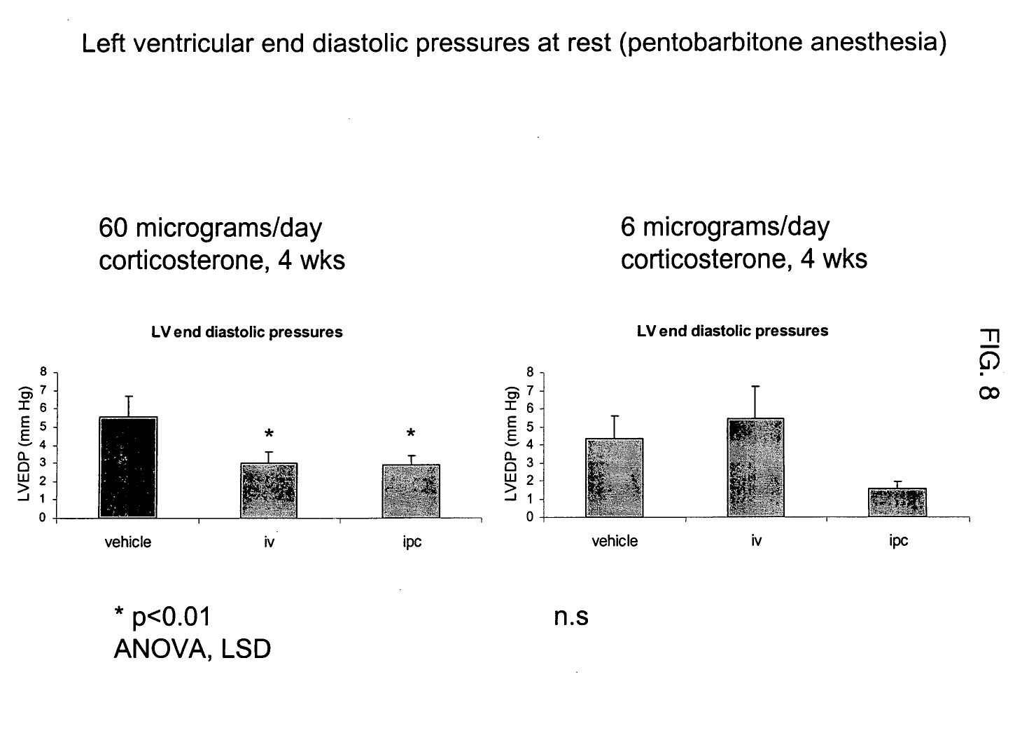

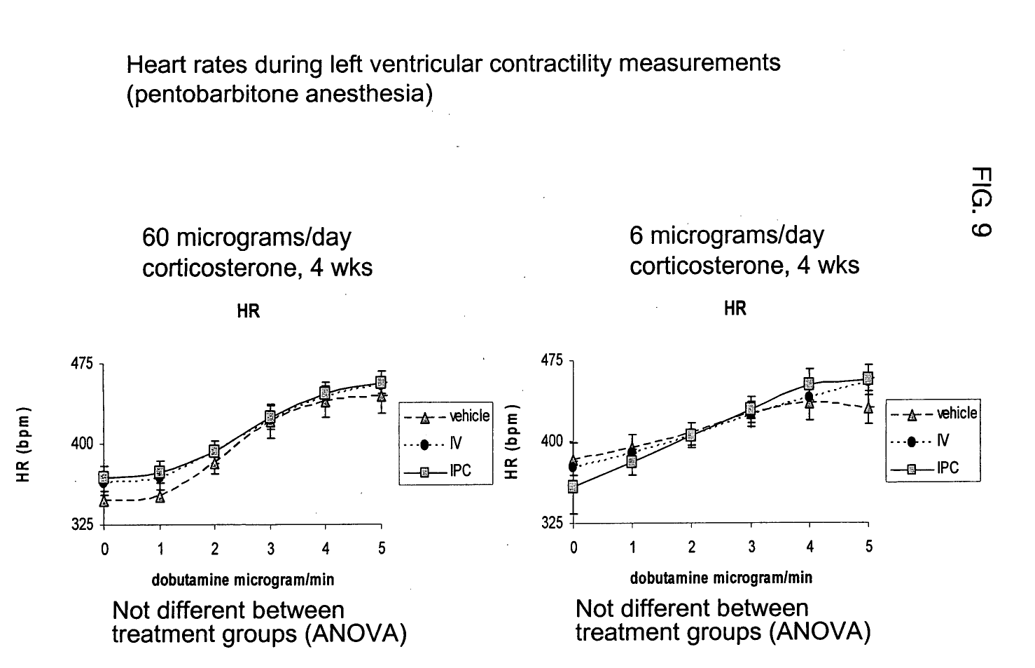

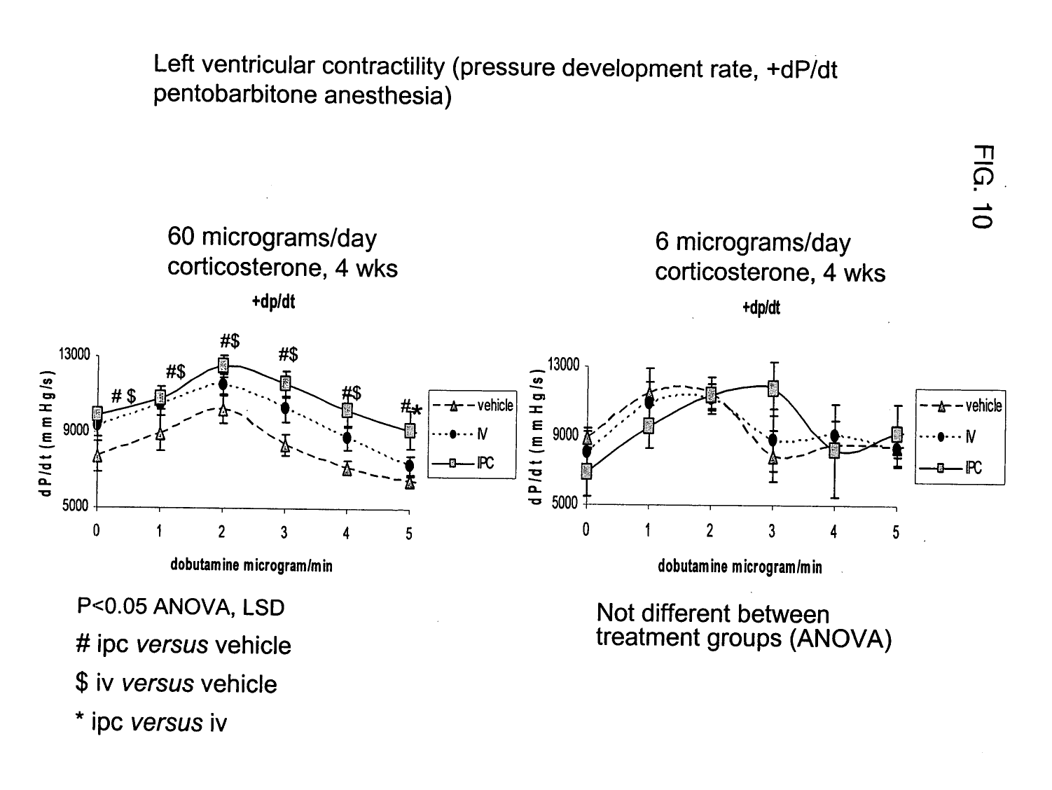

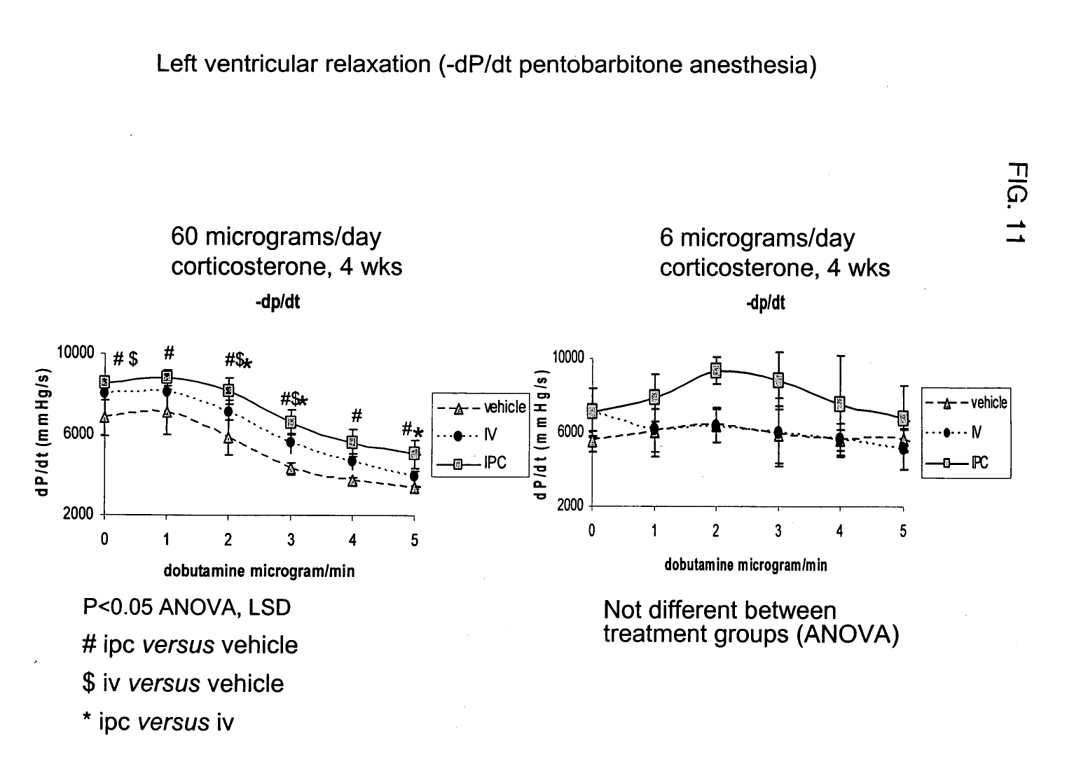

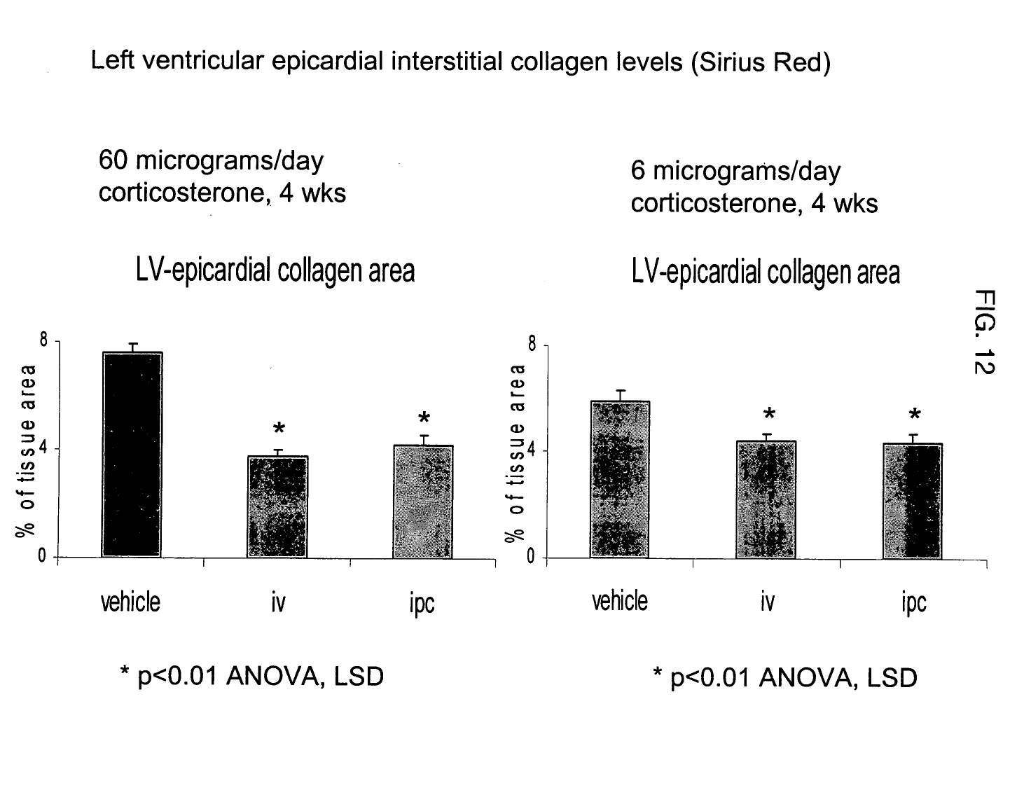

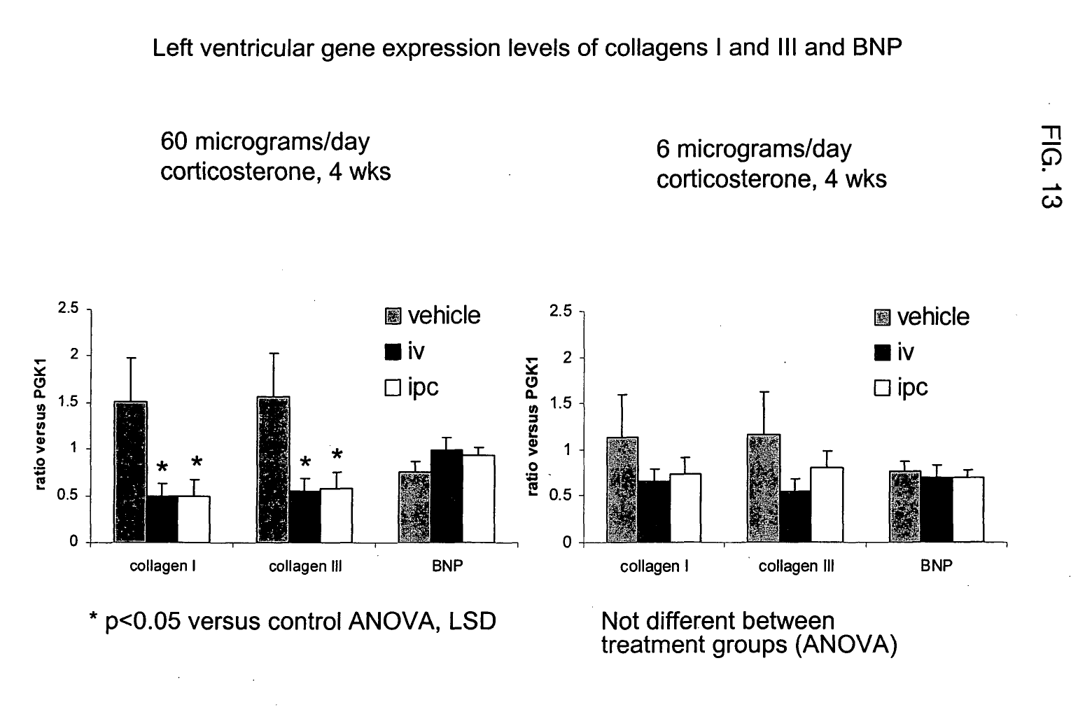

In Example 2, using the SHHF rat model of CHF it can be seen in a dose dependent fashion that corticosterone positively affects left ventricular contractility and relaxation, left ventricular end diastolic pressure, and left ventricular epicardial collagen area (see FIGS. 8, 9, 10, 11, and 12) while not affecting heart rate and blood pressure. In addition, corticosterone administration reduces mRNA expression of Type I and III collagen, and B-type natriuretic peptide (BNP) (see FIG. 13).

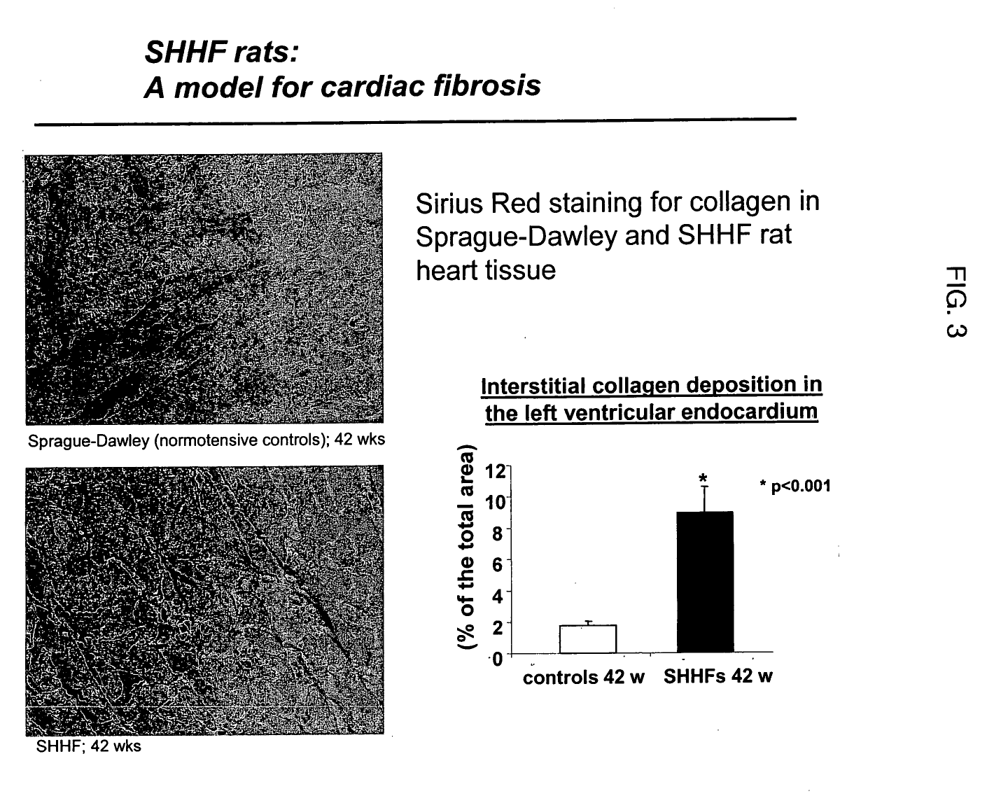

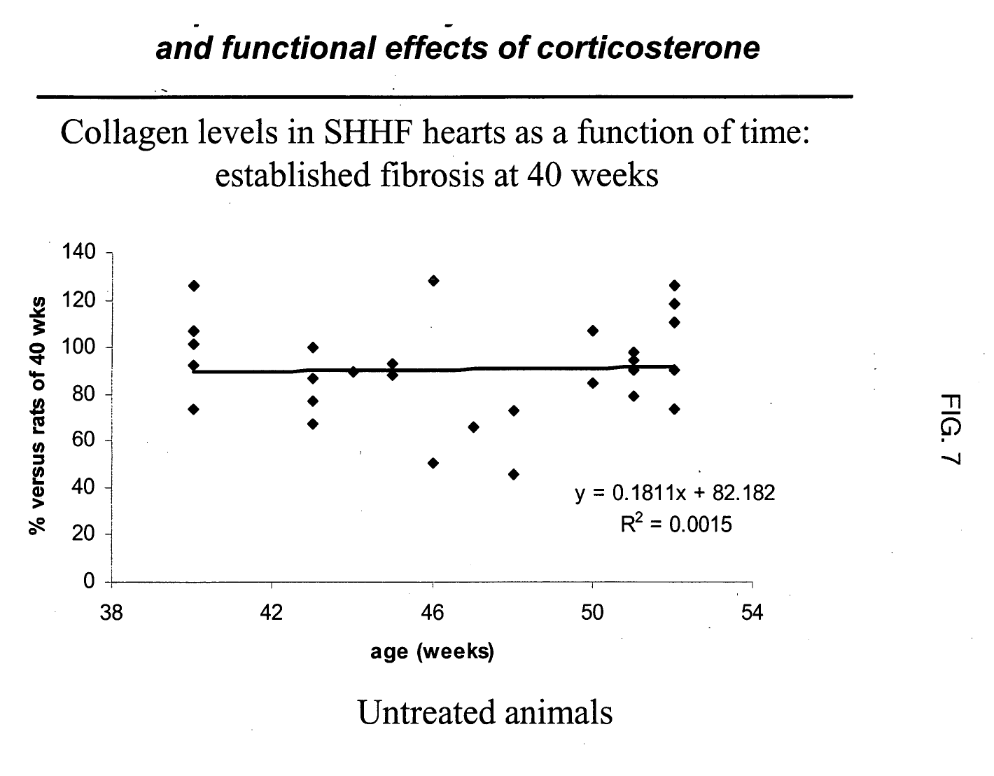

The SHHF rat model has been established as a model of CHF with predictive utility in screening compounds for safety and efficacy in humans. As demonstrated in FIGS. 3 and 7, elevated interstitial collagen levels are a histological and biochemical feature of aged (˜40 week) SHHF rats, a condition also observed in myocardial tissue from CHF patients. The data in FIGS. 3 and 7 was obtained generally using the procedure as described in Example 3. A reduction of interstitial collagen expression has been associated with improved cardiac function in SHHF rats and CHF patients.

Useful dosage forms and additional administration considerations are also disclosed herein.

The invention will now be described in more detail.

### B. DEFINITIONS

All percentages are weight percent unless otherwise noted.

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

The present invention is best understood by reference to the following definitions, the drawings and exemplary disclosure provided herein.

“Glucocorticoid(s)” means members of a class of steroid hormones (naturally occurring and synthetic) characterized by the ability to bind with the intracellular glucocorticoid receptor (GR), induce nuclear translocation of the GR-glucocorticoid complex and trigger similar effects on gene and protein expression. Glucocorticoids can be grouped into roughly three classes of compounds, based on the their relative potency as compared to cortisol typically defined as GR receptor binding affinity and/or activity in an in vitro or in vivo assay. Low potency glucocorticoids can be defined as those glucocorticoids having a potency from about 0.25 times to about 4 times the potency of cortisol. Examples of low potency glucocorticoids comprise: aclometasone, corticosterone, cortisol, cortisone acetate, desonide, hydrocortisone, or prednicarbate. Mid-potency glucocorticoids can be defined as those glucocorticoids having a potency from about 5 times to about 12 times the potency of cortisol. Examples of mid-potency glucocorticoids comprise: clobetasone, methylprednisolone, prednisone, prednisilone, or triamcinolone. High potency glucocorticoids can be defined as those glucocorticoids having a potency greater than or equal to about 13 times the potency of cortisol. Examples of high potency glucocorticoids comprise: amcinonide, betamethasone, beclomethasone, budenosid, clobetasol, desoximetasone, dexamethasone, diflorasone, fludrocortisones, fluocinonide, flurandrenolide, fluticasone, halcinonide, halobetasol, or mometasone. In embodiments, dosage forms according to the invention comprise at least one glucocorticoid in an amount ranging from about 0.0001 mg to about 1000 mg, preferably in an amount ranging from about 0.001 mg to about 500 mg, more preferably in an amount ranging from about 0.001 mg to about 100 mg, even more in an amount ranging from about 0.001 mg to about 50 mg, and yet more preferably in an amount ranging from about 0.01 mg to about 30 mg.

“Administering” or “administration” means providing a drug to a patient in a manner that is pharmacologically useful.

“Subject” is used interchangeably with “individual” and means any human with which it is desired to practice the present invention. The term “subject” does not denote a particular age, and the present systems are thus suited for use with subjects of any age, such as infant, adolescent, adult and senior aged subjects In certain embodiments, a subject may comprise a patient.

“Suffering from” means diagnosed with, or suspected as having, a particular medical condition.

“Congestive heart failure” means a structural or functional cardiac disorder that impairs the ability of the heart to fill with or pump a sufficient amount of blood throughout the body. Aspects of congestive heart failure mean the structural or functional changes due to congestive heart failure that can be observed clinically. In embodiments, aspects of congestive heart failure comprise: cardiac hypertrophy, reduced cardiac contractility, reduced cardiac output, pressure & volume overload hypertrophy, myocardial dysfunction, cardiac remodeling, post-myocardial infarction heart failure, and/or cardiopathy.

“Dosage form” means a composition suitable for pharmaceutical administration. Additional information regarding dosage forms useful in the practice of the invention is found elsewhere herein.

“Pharmaceutical carrier” means a pharmaceutically acceptable material that is pharmacologically inactive with respect to congestive heart failure. In embodiments, pharmaceutical carriers may comprise materials as described elsewhere herein.

“Systemic administration” means administration in a manner that provides for systemic absorption and distribution.

“Local administration” means administration in a manner that provides for absorption and distribution in a specific region or tissue of a subject.

### C. FURTHER DESCRIPTION

The doses of glucocorticoids useful in the practice of this invention may be varied depending upon the nature of the glucocorticoids, including such characteristics as potency and/or pharmacokinetic properties, and the condition of the subject.

Dosage forms according to the invention may be administered via a variety of routes. Routes of administration that are conventionally useful include systemic and local routes.

Systemic routes include but are not limited to: buccal, oral, inhalation, intravenous, intramuscular, nasal, transdermal, subcutaneous, and the like.

Administration of dosage forms according to the invention via local routes of administration may provide certain benefits versus systemic administration. For instance, as noted in J. J. R. Hermans et al., “The pharmacokinetic advantage of interpericardially applied substances in the rat” J Pharmacol Exp Ther 301:672-678 (2002) (“Hermans”), “Therapeutic efficacy may be limited because of difficulties in achieving satisfactory drug concentrations in the heart, whereas high systemic drug concentrations may lead to side effects.” As noted elsewhere in Hermans, certain forms of local administration, such as the intrapericardial administration described in Hermans, can provide enhanced local concentration of drugs while reducing the systemic concentration of the drug. Although Hermans fails to describe any administration of glucocorticoids to a subject suffering from congestive heart failure or related aspects thereof, Hermans' concept of local administration of drugs for pharmacokinetic and pharmacodynamic advantage can be usefully employed in the practice of the present invention.

Local routes include but are not limited to: interpericaridal, intrapericaridal, intramyocardial, intraperivascular, and interpericardium. Various methods for such local routes may be adapted for use in the practice of the present invention, including use of intravascular catheters, percutaneous injection, and the like.

Inventive dosage forms and methods may be used to administer glucocorticoids via a bolus administration or infusion. The choice of whether to use a bolus injection or an infusion may be based on a number of parameters, including the pharmacokinetic properties of the glucocorticoid, the condition of the subject, and the like.

A wide variety of dosage forms may be useful in the practice of this invention. Such dosage forms can be prepared using procedures well known in the pharmaceutical art. The dosage forms comprise a pharmaceutical carrier and at least one glucocorticoid. Pharmaceutical carriers may be utilized, in certain embodiments, to enhance stability of the at least one glucocorticoid, facilitate administration of the dosage forms, provide increased dissolution or dispersion, and the like. Information on pharmaceutical carriers useful in the practice of the present invention may be obtained from a variety of sources, including Remington: The Science and Practice of Pharmacy, 20th Edition, A. Gennaro (ed.), Lippincott Williams & Wilkins, 2000; Handbook of Pharmaceutical Additives, Michael & Irene Ash (eds.), Gower, 1995; Handbook of Pharmaceutical Excipients, A. H. Kibbe (ed.), American Pharmaceutical Ass'n, 2000; H. C. Ansel and N. G. Popovish, Pharmaceutical Dosage Forms and Drug Delivery Systems, 5th ed., Lea and Febiger, 1990.

As one of skill in the art would expect, the forms of the at least one glucocorticoid utilized in a particular pharmaceutical formulation are selected (e.g., salts) to possess suitable physical characteristics (e.g., water solubility) that are required for the formulation to be efficacious.

Dosage forms suitable for buccal (sub-lingual) administration include lozenges comprising at least one glucocorticoid in a flavored base, usually sucrose, and acacia or tragacanth, and pastilles comprising the at least one glucocorticoid in an inert base such as gelatin and glycerin or sucrose and acacia.

Dosage forms suitable for parenteral administration comprise sterile aqueous preparations of at least one glucocorticoid. These dosage forms are preferably administered intravenously, although administration can also be effected by means of subcutaneous, intramuscular, or intradermal injection, etc., and in accordance with the lists of routes of administration noted elsewhere herein. Injectable dosage forms are commonly based upon injectable sterile saline, phosphate-buffered saline, oleaginous suspensions, or other injectable carriers known in the art and are generally rendered sterile and isotonic with the blood. The injectable dosage forms may therefore be provided as a sterile injectable solution or suspension in a nontoxic parenterally acceptable diluent or solvent, including 1,3-butanediol, water, Ringer's solution, isotonic sodium chloride solution, fixed oils such as synthetic mono- or diglycerides, fatty acids such as oleic acid, and the like. Such injectable dosage forms are formulated using suitable dispersing or setting agents and suspending agents.

Solid dosage forms for oral administration of at least one glucocorticoid include capsules, tablets, pills, powders, and granules. For such oral administration, a dosage form containing at least one glucocorticoid is formed by the incorporation of any of the normally employed excipients, such as, for example, pharmaceutical grades of mannitol, lactose, starch, pregelatinized starch, magnesium stearate, sodium saccharine, talcum, cellulose ether derivatives, glucose, gelatin, sucrose, citrate, propyl gallate, and the like. Such solid dosage forms may include formulations, as are well known in the art, to provide prolonged or sustained delivery of the drug to the gastrointestinal tract by any number of mechanisms, which include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form.

Liquid dosage forms for oral administration of at least one glucocorticoid include emulsions, microemulsions, solutions, suspensions, syrups, and elixirs, optionally containing pharmaceutical adjuvants in a carrier, such as, for example, water, saline, aqueous dextrose, glycerol, ethanol and the like. These compositions can also contain additional adjuvants such as wetting, emulsifying, suspending, sweetening, flavoring, and perfuming agents.

Topical dosage forms that comprise at least one glucocorticoid include ointments, pastes, creams, lotions, gels, powders, solutions, sprays, inhalants, and aerosols, and may contain appropriate conventional additives such as preservatives, solvents to assist drug penetration and emollients in ointments and creams. Topical application may be once or more than once per day depending upon the usual medical considerations. Furthermore, glucocorticoids according to the invention can be administered in intranasal form via topical use of suitable intranasal dosage forms. The dosage forms may also contain compatible conventional carriers, such as cream or ointment bases and ethanol or oleyl alcohol for lotions. Such carriers may be present as from about 1% up to about 98% of the formulation, more usually they will form up to about 80% of the formulation.

Transdermal administration is also possible. Dosage forms suitable for transdermal administration can be presented as discrete patches adapted to remain in intimate contact with the epidermis of the recipient for a prolonged period of time. To be administered in the form of a transdermal delivery system, the dosage administration will, of course, be continuous rather than intermittent throughout the dosage regimen. Such patches suitably contain at least one glucocorticoid in an optionally buffered, aqueous solution, dissolved and/or dispersed in an adhesive, or dispersed in a polymer. A suitable concentration of the at least one glucocorticoids is about 0.01% to about 35 wt %, preferably about 0.01 wt % to about 15 wt %, based on the total weight of the dosage form.

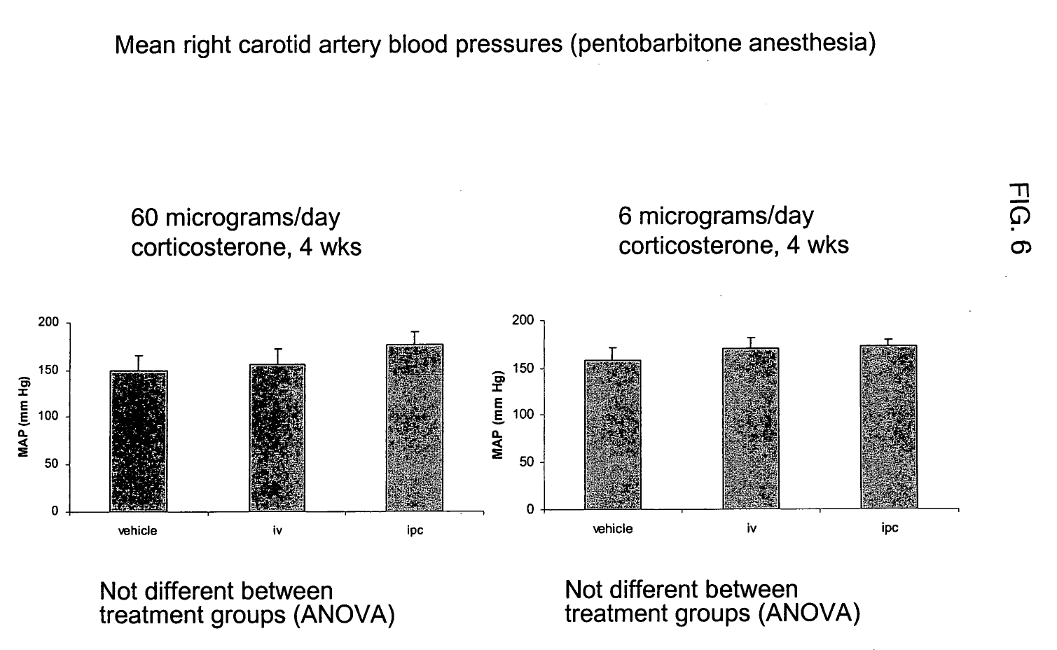

For administration by inhalation, the at least one glucocorticoid may be conveniently delivered in the form of an aerosol spray from a pump spray device not requiring a propellant gas or from a pressurized pack or a nebulizer with the use of a suitable propellant, e.g., dichlorodifluoromethane, trichlorofluoromethane, dichlorotetrafluoroethane, tetrafluoroethane, heptafluoropropane, carbon dioxide, or other suitable gas. In any case, the aerosol spray dosage unit may be determined by providing a valve to deliver a metered amount so that the resulting metered dose inhaler (MDI) is used to administer the at least one glucocorticoid in a reproducible and controlled way. Such inhaler, nebulizer, or atomizer devices are known in the art, for example, in PCT International Publication Nos. WO 97/12687 (particularly FIG. 6 thereof, which is the basis for the commercial RESPIMAT® nebulizer); WO 94/07607; WO 97/12683; and WO 97/20590.

Rectal administration can be effected utilizing unit dose suppositories in which the compound is admixed with low-temperature melting water-soluble or insoluble solids such as fats, cocoa butter, glycerinated gelatin, hydrogenated vegetable oils, mixtures of polyethylene glycols of various molecular weights, or fatty acid esters of polyethylene glycols, or the like. The at least one glucocorticoid may be a minor component, preferably from about 0.05 to about 10% by weight, with the remainder being the base component, based on total weight of the dosage form.

In all of the above dosage forms, the at least one glucocorticoid is formulated with an acceptable carrier or excipient. The carrier or excipient can be a solid or a liquid, or both, and is preferably formulated with the at least one glucocorticoid as a unit-dose composition, for example, a tablet, which can contain from 0.05% to 95% by weight of the active compound, based on the total weight of the dosage form. Such carriers or excipients include inert fillers or diluents, binders, lubricants, disintegrating agents, solution retardants, resorption accelerators, absorption agents, and coloring agents. Suitable binders include starch, gelatin, natural sugars such as glucose or beta.-lactose, corn sweeteners, natural and synthetic gums such as acacia, tragacanth or sodium alginate, carboxymethylcellulose, polyethylene glycol, waxes, and the like. Lubricants include sodium oleate, sodium stearate, magnesium stearate, sodium benzoate, sodium acetate, sodium chloride, and the like. Disintegrators include starch, methyl cellulose, agar, bentonite, xanthan gum, and the like. Pharmaceutically acceptable carriers encompass all the foregoing additives and the like.

While there has been described and pointed out features and advantages of the invention, as applied to present embodiments, those skilled in the medical art will appreciate that various modifications, changes, additions, and omissions in the method described in the specification can be made without departing from the spirit of the invention.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods within the scope of the invention, in addition to those enumerated herein, will be apparent to those skilled in the art from the foregoing description. Such modifications and variations are intended to fall within the scope of the appended claims. The present invention is to be limited only by the terms of the appended claims, along with the full scope of equivalents to which such claims are entitled.

The following Examples are meant to be illustrative of the claimed invention, and not limiting in any way.

### D. EXAMPLES

Data are expressed as mean±SEM. Statistical significance was assessed by one-way ANOVA followed by calculation of the LSD (least significant difference). Differences were considered significant at P<0.05

### Example 1

### Pharmacokinetic Study in Normal Wistar Rats

Normal Wistar rats were used for this Example. Animals were housed at the animal facilities of the University of Maastricht with a 12-hour light/dark cycle and had free access to standard rat chow and tap-water. Experiments were performed according to institutional guidelines and conformed to the Guide for the Care and Use of Laboratory Animals published by the US National Institutes of Health (NIH Publication No. 85-23, revised 1996).

The rats underwent a surgical procedure to install pericardial and intravenous catheters and minipumps. Construction of the pericardial catheters and the procedure to install the catheter into the pericardial space were conducted as described by Hermans, which has been discussed above.

Briefly, the pericardial catheters consisted of silicone tubing (internal diameter: 0.51 mm; external diameter 0.94 mm, Degania Silicone, Degania Bet, Israel), which, by assembling its endings with a polyolefin shrinking sleeve (Farnell Compounds, Maarssen, the Netherlands), was shaped as a loop (length appr. 2 cm, width appr. 1 cm). Silicone glue was applied in the middle of the loop, to create two separate chambers (one used for fluid injection, the second closed, i.e. connected to a closed ending). The fluid infusion chamber was provided with holes by use of a 25-gauge perforator. The endings of the silicone tubing were connected to (polyethylene) PE-10 tubing (i.d. 0.28 mm, e.d. 0.61 mm, Portex Limited, Kent, UK). Before installment, the catheter and the polyethylene extensions were gas sterilized and filled with sterile test solution before installment.

For pericardial catheter installment, rats were anesthetized by i.p. injection of 60 mg/kg sodium pentobarbitone (Nembutal, Sanofi Sante, Maassluis, the Netherlands) and placed on a heating pad, kept at 37° C. A high midline thoracotomy was performed and a retractor applied. Following careful cleavage of the sternohyoid muscle, thymus lobules were separated to expose the upper part of the pericardium. A small incision in the pericardial sac was made with iris scissors and the loop catheter inserted. The pericardial sac was closed by sealing it to the thymus with histoacryl tissue glue (Braun Melsungen Germany).

After removing the retractor, the polyethylene extensions were guided to the neck. Following a small loading bolus injection of 20 μl of sterile test-solution, the infusion extension was connected to a primed osmotic minipump (Alzet 2ml4, Durect, Cupertino, USA) filled with sterile test solution, using a small piece of PE-60. The closed extension was created by melting the end of the PE-10.

Intravenous catheters consisted of a tapered construction (filled with sterile test solution) of a PE-10 tip (hinged to enable insertion into the femoral vein and attachment), connected to PE-50, which in turn was connected to PE-60. With the rats still being under pentobarbitone anesthesia, the femoral vein was temporarily ligated, after which a small incision was made in the femoral vein with iris scissors. The catheter tip was inserted into the vein for about 4 cm to enter the vena cava, following fixation of the catheter with 3-0 silk and removal of the temporary ligation. The PE-60 part of the cannula was guided to the neck, following a small loading bolus injection with 100 μl of test-solution via the cannula, which was then connected to a primed osmotic minipump (Alzet 2ml4, Durect, Cupertino, USA) filled with sterile test solution. Osmotic minipumps (Alzet 2ml4, Durect, Cupertino, USA) were connected to the catheters and placed subcutaneously in the neck. These pumps provided a substantially constant infusion rate for at least 4 weeks.

All wounds were sutured with 3-0 silk (Ethicon, Norderstedt Germany). Animals were allowed to recover for 12 hours in a humidified environment at 37° C. before being transferred to the animal facilities of the University of Maastricht, where they were housed individually in their standard environment.

Total treatment time was 4 weeks for all animals. The rats were randomly assigned into 5 groups of 3-5 rats per group. The groups were as follows:
corticosterone high dose IPC (60 micrograms/day)
corticosterone low dose IPC (15 micrograms/day)
corticosterone high dose IV (60 micrograms/day)
corticosterone low dose IV (15 micrograms/day)
Control (vehicle)

Where IPC—intrapericardial dosing, and IV=intravenous dosing.

During treatment, Group 5 (Control) rats were continuously infused with vehicle (40% polyethylene glycol in water; 60 microliters/day) via both catheters. Experimental groups obtained continuous infusions as follows:

Group (1)—a high dose (60 ug/day) of corticosterone hemisuccinate intrapericardially and vehicle intravenously;
Group (2)—a low dose (15 ug/day) of corticosterone hemisuccinate intrapericardially and vehicle intravenously;
Group (3)—a high dose (60 ug/day) of corticosterone hemisuccinate intravenously and vehicle intrapericardially; and
Group (4)—a low dose (15 ug/day) of corticosterone hemisuccinate intravenously and vehicle intrapericardially.

Corticosterone hemisuccinate was purchased from Sigma (St Louis, Mo.).

At the end of the experiment, animals were anesthetized with pentobarbitone (60 mg/kg i.p.), following blood withdrawal from the aorta to sacrifice the animals and to be able to determine plasma levels of corticosterone. Pericardial fluid was collected and the hearts harvested following brief rinsing with saline. Hearts were cut in 3 slices and shock frozen. All samples were stored at −80° C. until analysed. For determining corticosterone levels, heart sections were weighed and homogenized in 5 volumes of 1 mM EDTA milliQ water. For plasma and pericardial fluid, corresponding amounts of 1 mM of EDTA were added. After addition of an internal standard, steroids were extracted with 12 volumes of diethylether, followed by transfer of the organic phase to conic tubes and evaporation of the solvent under a stream of nitrogen at 37° C. Steroids were collected in the cone of the tube by rinsing the tube twice with 0.2 ml of acetone and evaporation of the acetone. To the dry residues, 30 microliters of acetone were added, to re-dissolve the steroids. Then, 60 microliters of sulphuric acid (98%) were added to convert the steroids into fluorescent derivatives. Steroids were allowed to react for 20 minutes in the dark. Subsequently, 6 ml of diethylether was added, along with 0.5 ml of water and the steroids were extracted into the organic phase in order to remove the sulphuric acid. After transfer of the organic phase into tubes, solvent was evaporated under a stream of nitrogen at 37° C. Samples were reconstituted in mobile phase and subjected to HPLC analysis. The HPLC system consisted of a Varian Inertsil ODS-3 100×3 mm as stationary phase and acetonitrile/methanol/milliQ/Trifluoroacetic acid=250/360/360/1 v/v/v/v as mobile phase (pumped at 0.8 ml/min). Detection was fluorescence at excitation wavelength 367 nm emission 532 nm (at Shimadzu RF-10Axl). Peak areas of corticosterone and internal standard were calculated and ratios were compared with a calibration curve to calculate corticosterone concentrations of the samples.

The results are shown in FIGS. 1-2.

### Example 2

### Study of Aspects of CHF in Spontaneous Hypertensive Heart Failure Rats after IPC or IV Administration of Corticosterone

Forty to forty-two week old male lean spontaneous hypertensive heart failure (SHHF) rats (originally obtained from Charles River, Boston, USA) were used. Animals were housed at the animal facilities of the University of Maastricht with a 12-hour light/dark cycle and had free access to standard rat chow and tap-water. Experiments were performed according to institutional guidelines and conformed to the Guide for the Care and Use of Laboratory Animals published by the US National Institutes of Health (NIH Publication No. 85-23, revised 1996).

The rats underwent a surgical procedure to install pericardial and intravenous catheters and minipumps according to the procedure of Example 1.

Animals were randomly assigned to treatment groups (n=9 per group). Total treatment time was 4 weeks for all animals. The groups were as follows:
(1) corticosterone high dose IPC (60 micrograms/day)
(2) corticosterone low dose IPC (6 micrograms/day)
(3) corticosterone high dose IV (60 micrograms/day)
(4) corticosterone low dose IV (6 micrograms/day)
(5) vehicle

Where IPC—intrapericardial dosing, and IV=intravenous dosing.

During treatment, Group 5 (vehicle) rats were continuously infused with vehicle (40% polyethylene glycol in water; 60 μl/day) via both catheters. Experimental groups obtained continuous infusions as follows:
Group (1)—a high dose (60 ug/day) of corticosterone hemisuccinate intrapericardially and vehicle intravenously;
Group (2)—a low dose (6 ug/day) of corticosterone hemisuccinate intrapericardially and vehicle intravenously;
Group (3)—a high dose (60 ug/day) of corticosterone hemisuccinate intravenously and vehicle intrapericardially; and
Group (4)—a low dose (6 ug/day) of corticosterone hemisuccinate intravenously and vehicle intrapericardially.
Corticosterone hemisuccinate was purchased from Sigma (St Louis, Mo.)

Left ventricular pressures, contractility and relaxation were evaluated at the end of the study as described in B J Janssen et al., “Effects of anesthetics on systemic hemodynamics in mice.” Am J Physiol Heart Circ Physiol 287:H1618-1624 (2004). For this purpose rats were anaesthetized with pentobarbital (50 mg/kg i.p., Sigma). A high-fidelity catheter tip micromanometer (Mikro-tip 1.4F; SPR-671, Millar Instruments, Houston, Tex., USA) was inserted into the left ventricular cavity through the right carotid artery. Before the tip was inserted into the left ventricle, mean arterial pressures and heart rates were briefly recorded in the carotid artery. Left ventricular pressure was measured and sampled at a rate of 2 kHz under an I.V. ramp infusion of dobutamine up to 5 ng/gBW/min (Sigma) using a microinjection pump (model 200 Series, KD Scientific, Boston, Mass., USA). Maximal positive (+dp/dt/p) and negative (−dp/dt/p) pressure development were determined. At the end of the experiment blood and organs were harvested, weighed and rapidly processed and frozen for future analyses.

Hearts were harvested for histological analysis in order to determine fibrillar interstitial and perivascular collagen accumulation by collagen specific Picro Sirius Red staining and subsequent light microscopy. For this purpose, the apical parts of the hearts were embedded in Tissue-tek and frozen in freezing 2-methyl-butane in liquid nitrogen. Cryostat cross-sections (6 μm) were prepared, air dried, fixed in 10% buffered formalin for 1 hour and washed for 30 min followed by 5 min in distilled water. After 5 min in 0.2% Phosphomolybdic acid for prevention of background staining all sections were stained for 90 min with 0.1% Sirius red (Polyscience, Warrington Pa., USA) in saturated picric acid. Before coverslipping with Entelan all sections were rapidly dehydrated by subsequent sequential dipping in 70 v/v %, 96 v/v ethanol/milliQ, and 100% ethanol, followed by xylene.

Collagen volume fraction of each section was determined using a computer image analyzing system (Leica Q-Win). Interstitial collagen density of heart sections was evaluated in the left ventricle (LV) subepicardial myocardium regions. Ten fields from each region (magnification ×20) were randomly selected for analyses and the area stained was calculated as percentage of the total area within a field.

Perivascular collagen was expressed as ratio of the area of the collagen surrounding the vessel divided to the area of the coronary arteries media in order to correct for differences in vessel size (magnification ×20 or ×40). We calculated an average value for interstitial and perivascular fibrosis or each animal.

All analyses were performed in a blinded fashion.

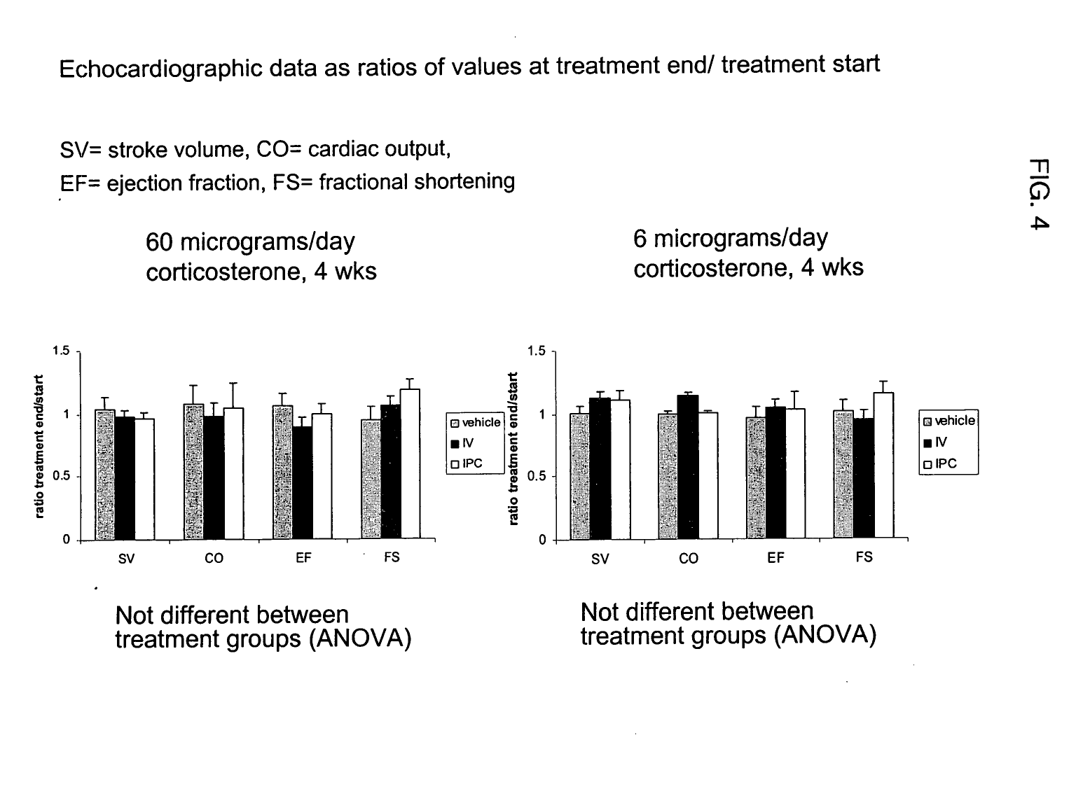

At the start and end of the treatment, transthoracic echocardiography was performed under isoflurane anesthesia in part of the rats (5-7 per group) using the SONOS 5500 echocardiographic system equipped with a 15-MHz linear-array transducer (Philips Medical Systems Corp., Netherlands). In brief, rats were anesthetized with 2-3% isoflurane (Abbott Laboratories, Kent, UK) placed on a heating pad, kept at 37° C. with transducer placed on the left hemithorax. The 2-dimensional parasternal long- and short-axis view of the left ventricle and the parasternal short axis M-mode tracings were recorded. Results of the echocardiography are shown in FIG. 4.

Measurements and calculations performed are the following: percent LV fractional shortening (FS) was calculated as follows: FS=(LVIDd LVIDs)/LVIDd×100, where LVIDd and LVIDs are end-diastolic and end-systolic left ventricle internal dimensions, respectively. End-diastolic (EDV) and end-systolic volumes (ESV) were calculated from left ventricle systolic (LVAs) and diastolic (LVAd) areas via the method of discs. Ejection fraction (EF) was calculated from systolic and diastolic volumes with the following formula: EF=(EDV ESV)/EDV×100. Other measurements taken include heart rate (HR-derived from the m-mode by determining the of RR interval; m-mode R-R interval), stroke volume (SV; SV=EDV ESV) and cardiac output (CO=SV×HR).

No effects of the treatments were seen on left ventricular dimensions or performance parameters.

Left ventricular gene expression levels of collagens I and III and BNP were determined as follows. mRNA expression analyses were performed as follows:

Heart samples (i.e. basal side of the hearts) were shock frozen in liquid nitrogen for RNA. Total RNA from the LV was isolated using the Ultraspec II kit (Bioteckx), according to the instructions of the manufacturer. After integrity checking and determining RNA concentrations with the nanodrop procedure, the RNA concentrations were adjusted to 100 ng/μl For first strand cDNA synthesis, 100 ng heat denatured RNA and 200 pmol random hexamer primers (Pharmacia, Uppsala Sweden) were transferred to a Ready-To-Go first strand bead (Pharmacia, Uppsala Sweden) tube and the volume adjusted to 33 μl.

The reaction (1 hour at 37° C.) was stopped by heat inactivating (2 minutes at 94° C.) the transcriptase and samples were cooled to 4° C. For conducting the real-time PCR, 10 ml of the cDNA templates (100× diluted) were added to SYBR green master mix (Eurogentec) and primers (15 μM final concentration each) yielding a total volume of 25 ml, following the instructions of the Eurogentec.

PCR-primers (Sigma Genosys) were selected based on published primers/cDNA sequences and had the following sequences (5′→3′):

CGGAGACACCGCCACTTG
(5′-primer PGK 1),
AAGGCAGGAAAATACTAAACAT
(3′-primer PGK 1),
GCTGCTTTGGGCAGAAGATAGA
(5′-primer brain natriuretic peptide (BNP)),
ACAACCTCAGCCCGTCACA
(3′-primer BNP),
CGAAGGCAACAGTCGATTCA
(5′-primer procollagen (α1) type I),
GGTCTTGGTGGTTTTGTATTCGAT
(3′-primer procollagen (α1) type I),
TCCTGAAGATGTCCTTTGATGTACAG
(5′-primer procollagen (α1) type III)
and
TTCAGAGACTTCTTTACATTGCCATT
(3′-primer procollagen (α1) type III),

yielding products with fragment lengths of 253, 147 and 86 base pairs for PGK 1 (phosphoglycerate kinase 1), BNP (brain natriuretic peptide) and collagens I and -III respectively.

Samples were denatured and incubated for 40 cycles at 60° C. (120 seconds; annealing and synthesis) and 95° C. (15 seconds), during which fluorescence was continuously monitored (Biorad thermocycler).

Based on the linear part of the curves, initial gene concentrations were derived by comparing signals with standard curves. Validity of the procedure was checked by constructing melting curves for every sample.

Calculated gene expression levels were standardized for the house keeping gene PGK1 and were normalized versus RNA obtained from untreated control animals. Data are therefore expressed as arbitrary units (means±SEM).

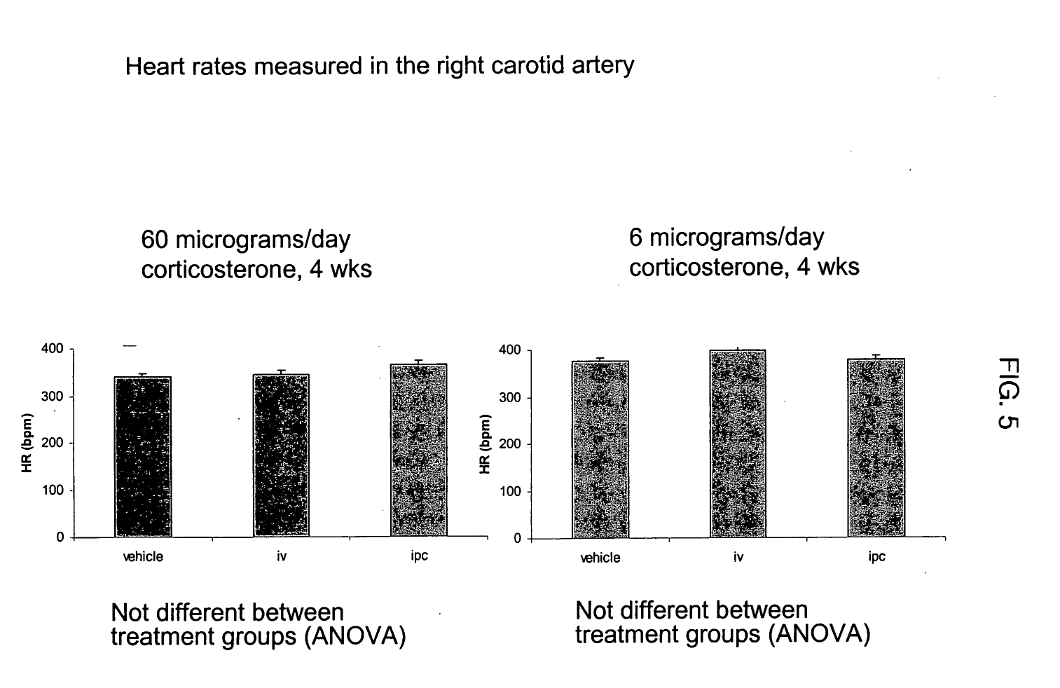

Results are shown in FIGS. 4-6, and 8-13.

### Example 3

### Collagen Deposition Comparisons

Male Sprague-Dawley and male lean spontaneous hypertensive heart failure (SHHF) rats and (originally obtained from Charles River, Boston, USA) were used. Animals were housed at the animal facilities of the University of Maastricht with a 12-hour light/dark cycle and had free access to standard rat chow and tap-water. Experiments were performed according to institutional guidelines and conformed to the Guide for the Care and Use of Laboratory Animals published by the US National Institutes of Health (NIH Publication No. 85-23, revised 1996).

In order to characterize the extent of cardiac fibrosis (collagen deposition) in SHHF rats, when compared with normal (Sprague-Dawley) rats, 4 SHHF rats and 8 Sprague-Dawley rats were sacrificed under pentobarbital anesthesia (60 mg/kg i.p.) at the age of 42 weeks and their hearts harvested for histology analysis of left ventricular collagen as described below.

Furthermore, to evaluate whether the amount of cardiac collagen is changing with age, a total number of 28 SHHF rats was sacrificed under pentobarbital anesthesia (60 mg/kg i.p.) between the age of 39 and 52 weeks.

Hearts were harvested for histological analysis in order to determine fibrillar interstitial and perivascular collagen accumulation by collagen specific Picro Sirius Red staining and subsequent light microscopy. For this purpose, the apical parts of the hearts were embedded in Tissue-tek and frozen in freezing 2-methyl-butane in liquid nitrogen. Cryostat cross-sections (6 μm) were prepared, air dried, fixed in 10% buffered formalin for 1 hour and washed for 30 min followed by 5 min in distilled water. After 5 min in 0.2% Phosphomolybdic acid for prevention of background staining all sections were stained for 90 min with 0.1% Sirius red (Polyscience, Warrington Pa., USA) in saturated picric acid. Before coverslipping with Entelan all sections were rapidly dehydrated by subsequent sequential dipping in 70 v/v %, 96 v/v ethanol/milliQ, and 100% ethanol, followed by xylene.

Collagen volume fraction of each section was determined using a computer image analyzing system (Leica Q-Win). Interstitial collagen density of heart sections was evaluated in the left ventricle (LV) subepicardial myocardium regions. Ten fields from each region (magnification ×20) were randomly selected for analyses and the area stained was calculated as percentage of the total area within a field.

All analyses were performed in a blinded fashion.

Statistical analysis of the differences between SHHF rats and Sprague-Dawley rats was by t-test. Possible age-dependency of the LV collagen content in SHHF rats was assessed by linear regression analysis (least squares method).

Results (FIGS. 3 and 7) clearly show that 42 weeks old SHHF rats display extensive collagen deposition, when compared with normotensive Sprague-Dawley rats, whereas LV collagen levels in SHHF rats do not change between 39 and 52 weeks of age.

### Example 4

### Tablet Dosage Form

### Prophetic

Component Amount per tablet (in mg):

| | |
|---|---|
| Desonide | 100 |
| Lactose | 140 |
| corn starch | 240 |
| polyvinylpyrrolidone | 15 |
| magnesium stearate | 5 |
| TOTAL | 500 |

The finely ground desonide, lactose, and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

### Example 5

### Tablet Dosage Form

### Prophetic

Component Amount per tablet (in mg):

| | |
|---|---|
| aclometasone | 80 |
| lactose | 55 |
| corn starch | 190 |
| polyvinylpyrrolidone | 15 |
| magnesium stearate | 2 |
| microcrystalline cellulose | 35 |
| sodium-carboxymethyl starch | 23 |
| TOTAL | 400 |

The finely ground aclometasone, some of the corn starch, lactose, microcrystalline cellulose, and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

### Example 6

### Coated Tablet Dosage Form

### Prophetic

Component Amount per tablet (in mg):

| | |
|---|---|
| prednisone | 5 |
| lactose | 30 |
| corn starch | 41.5 |
| polyvinylpyrrolidone | 3 |
| magnesium stearate | 0.5 |
| TOTAL | 90 |

The prednisone, corn starch, lactose, and polyvinylpyrrolidone are thoroughly mixed and moistened with water. The moist mass is pushed through a screen with a 1 mm mesh size, dried at about 45.degree. C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

### Example 7

### Capsule Dosage Form

### Prophetic

Component Amount per capsule (in mg):

| | |
|---|---|
| clobetasone | 50 |
| corn starch | 268.5 |
| magnesium stearate | 1.5 |
| TOTAL | 320 |

The clobetasone and corn starch are mixed and moistened with water. The moist mass is screened and dried. The dry granules are screened and mixed with magnesium stearate. The finished mixture is packed into size 1 hard gelatine capsules.

### Example 8

### Ampoule Solution Dosage Form

### Prophetic

Component Amount per ampoule:

| | |
|---|---|
| prednisilone | 50 mg |
| sodium chloride | 50 mg |
| water for inj. | 5 mL |

The prednisilone is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilized and sealed by fusion. The ampoules contain 5 mg, 25 mg, and 50 mg of active prednisilone as desired.

### Example 9

### Aerosol Dosage Form

### Prophetic

### Component Amount

| | |
|---|---|
| beclomethasone | 0.005% |
| sorbitan trioleate | 0.1% |
| monofluorotrichloromethane and | to 100% |
| difluorodichloromethane (2:3) | |

The suspension is transferred into a conventional aerosol container with a metering valve. Preferably, 50 microliters of suspension are delivered per spray. The beclomethasone may also be metered in higher doses if desired (e.g., 0.02% by weight).

### Example 10

### Local Administration of Glucocorticoid

### Prophetic

Clobetasol is combined with sterile Ringer's solution to form a sterile clobetasol solution having a concentration of 0.01 wt % clobetasol.

Intrapericardial fluid communication is established in a subject according to the teachings of United States Published Patent Application 2006/0229492 to Gelfand et al. (“Gelfand”). Instead of introducing the injectable heart constrainer of Gelfand, the clobetasol solution is infused at a rate sufficient to provide 0.25 mg of clobetasol in the pericardial space. The introducing catheter is then withdrawn, and the incisions closed, again according to the teachings of Gelfand, and coupled with standard surgical techniques.

What is claimed is:

1. A method comprising:
administering to a subject suffering from congestive heart failure a dosage form comprising a pharmaceutical carrier and at least one glucocorticoid in an amount ranging from about 0.0001 mg to about 1000 mg.

2. The method of claim 1, wherein the glucocorticoid comprises aclometasone, corticosterone, cortisol, cortisone acetate, desonide, hydrocortisone, prednicarbate, clobetasone, methylprednisolone, prednisone, prednisilone, triamcinolone, amcinonide, betamethasone, beclomethasone, budenosid, clobetasol, desoximetasone, dexamethasone, diflorasone, fludrocortisones, fluocinonide, flurandrenolide, fluticasone, halcinonide, halobetasol, or mometasone.

3. The method of claim 1, wherein the glucocorticoid is a low potency glucocorticoid.

4. The method of claim 1, wherein the glucocorticoid is a high potency glucocorticoid.

5. The method of claim 1, wherein the dosage form comprises at least one glucocorticoid in an amount ranging from about 0.001 mg to about 500 mg.

6. The method of claim 5, wherein the dosage form comprises at least one glucocorticoid in an amount ranging from about 0.001 mg to about 100 mg.

7. The method of claim 6, wherein the dosage form comprises at least one glucocorticoid in an amount ranging from about 0.001 mg to about 50 mg.

8. The method of claim 7, wherein the dosage form comprises at least one glucocorticoid in an amount ranging from about 0.01 mg to about 30 mg.

9. The method of claim 1, wherein the glucocorticoid is administered systemically.

10. The method of claim 1, wherein the glucocorticoid is administered locally to cardiac tissue.

11. The method of claim 1, wherein the glucocorticoid is administered by infusion.

12. The method of claim 1, wherein the congestive heart failure comprises aspects that comprise at least one of cardiac hypertrophy, reduced cardiac contractility, reduced cardiac output, pressure & volume overload hypertrophy, myocardial dysfunction, cardiac remodeling, post-myocardial infarction heart failure, or cardiopathy.

13. A method comprising:
administering a dosage form comprising a pharmaceutical carrier and at least one glucocorticoid to a subject suffering from congestive heart failure, wherein the at least one glucocorticoid is present in the dosage form in an amount effective to ameliorate aspects of the congestive heart failure.

14. The method of claim 13, wherein the aspects of the congestive heart failure comprise at least one of cardiac hypertrophy, reduced cardiac contractility, reduced cardiac output, pressure & volume overload hypertrophy, myocardial dysfunction, cardiac remodeling, post-myocardial infarction heart failure, or cardiopathy.

15. The method of claim 13, wherein the at least one glucocorticoid comprises aclometasone, corticosterone, cortisol, cortisone acetate, desonide, hydrocortisone, prednicarbate, clobetasone, methylprednisolone, prednisone, prednisilone, triamcinolone, amcinonide, betamethasone, beclomethasone, budenosid, clobetasol, desoximetasone, dexamethasone, diflorasone, fludrocortisones, fluocinonide, flurandrenolide, fluticasone, halcinonide, halobetasol, or mometasone.

16. The method of claim 13, wherein the glucocorticoid is a low potency glucocorticoid.

17. The method of claim 13, wherein the glucocorticoid is a high potency glucocorticoid.

18. The method of claim 13, wherein the dosage form comprises at least one glucocorticoid in an amount ranging from about 0.0001 mg to about 1000 mg.

19. The method of claim 18, wherein the dosage form comprises at least one glucocorticoid in an amount ranging from about 0.001 mg to about 500 mg.

20. The method of claim 19, wherein the dosage form comprises at least one glucocorticoid in an amount ranging from about 0.001 mg to about 100 mg.

21. The method of claim 20, wherein the dosage form comprises at least one glucocorticoid in an amount ranging from about 0.001 mg to about 50 mg.

22. The method of claim 21, wherein the dosage form comprises at least one glucocorticoid in an amount ranging from about 0.01 mg to about 30 mg.

23. The method of claim 13, wherein the glucocorticoid is administered systemically.

24. The method of claim 13, wherein the glucocorticoid is administered locally to cardiac tissue.

25. The method of claim 13, wherein the glucocorticoid is administered by infusion.
